(19) United States
(12) Patent Application Publication
(10) Pub. No.: US 2017/0181733 A1
(43) Pub. Date: Jun. 29, 2017

(54) ENDOVASCULAR CYTOLOGY BRUSH AND METHOD FOR USE

(71) Applicant: BRIGHAM AND WOMEN'S HOSPITAL, INC., Boston, MA (US)

(72) Inventors: Jane A. Leopold, Chestnut Hill, MA (US); Bradley A. Maron, Sharon, MA (US); Aaron B. Waxman, Newton, MA (US); Jospeh Loscalzo, Dover, MA (US)

(21) Appl. No.: 15/309,522

(22) PCT Filed: May 7, 2015
(86) PCT No.: PCT/US15/29672

§ 371 Date: Nov. 8, 2016

Related U.S. Application Data
(60) Provisional application No. 61/990,754, filed on May 9, 2014.

(51) Int. Cl. A61B 10/02

(52) U.S. Cl. CPC A61B 10/02; A61B 10/0233; A61B 2010/0216; A61B 10/04

(57) ABSTRACT

Systems and methods directed to sampling endothelial cells within a pulmonary vasculature are provided. The system and method may include a cell sampling device having an elongated sheath extending from a proximal end to a distal end of the cell sampling device and defining an access lumen. An endovascular brush includes brush elements coupled to a shaft. The endovascular brush is dimensioned to be received in the access lumen of the elongated sheath. An actuation control is coupled to the shaft of the endovascular brush to axially translate the endovascular brush between a retracted position and an expanded position. Upon translation of the endovascular brush from the retracted position to the expanded position, the brush elements moves from within the access lumen to the pulmonary vasculature to obtain endothelial cells from a target artery of the pulmonary vasculature.

### CROSS-REFERENCE TO RELATED APPLICATIONS

This application is based on, claims the benefit of, and incorporates herein by reference, U.S. Provisional Patent Application Ser. No. 61/990,754, filed on May 9, 2014 and entitled “Vascular Cytology Brush.”

### STATEMENT REGARDING FEDERALLY SPONSORED RESEARCH

NA

### BACKGROUND

The present disclosure relates generally to systems and methods for an endovascular cytology brush for use in the pulmonary vasculature to sample endothelial cells. More particularly, the disclosure relates to systems and methods for an endovascular cytology brush adapted to the pulmonary vasculature and used to isolate pulmonary vascular endothelial cells for diagnosis, prognosis, selecting a therapy, or evaluating a response therapy.

Pulmonary arterial hypertension (PAH) is an insidious disease associated with pulmonary vascular remodeling that promotes right heart failure and premature death. In PAH, pulmonary endothelial dysfunction is identified as a critical mediator of distal pulmonary arteriole remodeling to induce pulmonary hypertension. To date, the spectrum of pulmonary endothelial dysfunction that is present in pre-clinical, clinically-relevant, and drug-treated pulmonary hypertension remains unknown, as does the relationship between endothelial dysfunction and increased pulmonary pressures, pulmonary vascular resistance, and right ventricular remodeling. Similarly, while contemporary pharmacotherapies for PAH may improve symptoms by replacing deficient endothelial-derived products (e.g., nitric oxide, prostacyclin, etc.) to the pulmonary vasculature, strategies for monitoring dynamic changes in the pulmonary endothelial phenotype in patients are not available.

Although the etiology of PAH may vary between patients, the cardiac and pulmonary vascular pathophenotypes are remarkably similar. PAH is characterized by aberrant vascular remodeling with distal pulmonary artery endothelial dysfunction and dysregulated vascular smooth muscle cell proliferation leading to inflammation, neointimal formation, thrombosis, and luminal obliteration. The formation of plexiform lesions is progressive and contributes to impaired vascular reactivity, elevated pulmonary artery pressure, and increased pulmonary vascular resistance, which, in turn, promotes maladaptive right ventricular remodeling. The burden of structural and functional disruption of the pulmonary vascular-right ventricular circuit ultimately leads to right ventricular failure and death.

PAH is associated with high rates of morbidity and mortality with approximately 45% mortality within 3 years of diagnosis, despite optimal medical therapy. To date, there are no peripheral biomarkers or diagnostic tests that identify preclinical PAH; provide prognostic information based on indices of pulmonary vascular remodeling; allow for the selection of tailored therapy based on genetic profiling, similar to cancer therapeutics; or monitor current disease status and response to medications. This deficit has occurred owing to the inaccessibility of the pulmonary vascular compartment for tissue or vascular cell sampling of endothelial cells. The vascular remodeling changes and endothelial dysfunction described in PAH have also been described, in part, for other forms of pulmonary hypertension.

The endothelium, the cells that line the luminal surface of blood vessels, sense the micro-milieu of flowing blood and regulate important functions of blood vessels. For example, endothelial cells synthesize potent vasodilators and constrictors to alter the vascular tone in order to control blood pressure and blood flow. Endothelial cells also produce cytokines that mediate inflammatory responses in the vessel wall, which underscores the pathogenesis of many vascular diseases, such as atherosclerosis. Accordingly, a number of drugs have been developed for treating vascular diseases which target the endothelium. Endothelial gene expression changes in response to drug therapy long before anatomical changes or clinical symptoms occur. It is therefore possible to use endothelial gene expression patterns as a surrogate indicator of drug efficacy to facilitate drug design and testing.

A number of brushes have been adopted in the art for minimally invasive cell sampling, such as the biopsy brushes designed for use in obtaining biopsy samples from the urinary, tracheal, bronchial, and gastrointestinal tracts. However, these brushes are not suitable for endovascular use, particularly with respect to relatively small blood vessels. Additionally, the brushes are often not very effective in obtaining cell samples.

Accordingly, there is a need for an endovascular brush capable of being used in endovascular procedures. There is also a need for an endovascular brush that is capable of being used for endovascular cell sampling, including in-vivo endovascular cell sampling.

### SUMMARY

The present disclosure overcomes the aforementioned drawbacks by providing a system and method for an endovascular cytology brush capable of being utilized for repeated sampling of pulmonary artery endothelial cells for endothelial phenotypic profiling in PAH and other forms of pulmonary hypertension (World Health Organization Groups 1-5). The endothelial phenotypic profiling may use gene expression microarrays to reveal endothelial pathophenotypes that can serve as a biomarker of PAH disease severity. These profiles may also be exploited for the selection of drug therapies or to identify druggable targets. Thus, characterization of the cells may identify endothelial disease-related profiles that can provide diagnostic and prognostic information. Use of the disclosed endovascular cytology brush has broad applicability to patients with other pulmonary diseases where knowledge of the endothelial cell phenotype aides in prognosis, diagnosis, or therapeutic choices, such as following pulmonary transplant. In addition, the endovascular cytology brush may be applied to patients with lung pathologies, such as rejection in lung transplantation or inflammatory lung diseases.

In accordance with one aspect of the disclosure, a cell sampling device for sampling endothelial cells within a pulmonary vasculature is provided. The cell sampling device includes an elongated sheath extending from a proximal end to a distal end of the cell sampling device and defining an access lumen. An endovascular brush includes brush elements coupled to a shaft. The endovascular brush is dimensioned to be received in the access lumen of the elongated sheath. An actuation control is coupled to the shaft of the endovascular brush to axially translate the endovascular brush between a retracted position and an expanded position. Upon translation of the endovascular brush from the retracted position to the expanded position, the brush elements moves from within the access lumen to the pulmonary vasculature to obtain endothelial cells from a target artery of the pulmonary vasculature.

In accordance with another aspect of the disclosure, a method for sampling endothelial cells within a pulmonary vasculature, is provided. The method includes inserting an elongated sheath into the pulmonary vasculature of a subject, the elongated sheath defining an access lumen. An endovascular brush is inserted into the access lumen of the elongated sheath. The endovascular brush includes brush elements coupled to a shaft. An actuation control coupled to the shaft of the endovascular brush is used to axially translate the endovascular brush from a retracted position within the access lumen to an expanded position in a target artery of the pulmonary vasculature. Using the brush elements of the endovascular brush, endothelial cells from the target artery are obtained to determine a state of the pulmonary vasculature.

The foregoing and other aspects and advantages of the disclosure will appear from the following description. In the description, reference is made to the accompanying drawings which form a part hereof, and in which there is shown by way of illustration a preferred embodiment of the disclosure. Such embodiment does not necessarily represent the full scope of the disclosure, however, and reference is made therefore to the claims and herein for interpreting the scope of the disclosure.

### BRIEF DESCRIPTION OF THE DRAWINGS

FIG. 1A is a side view of a cell sampling device according to one embodiment of the present disclosure.

FIG. 1B is an enlarged view of an endovascular brush of the cell sampling device of FIG. 1A.

FIG. 2 is a side cross-sectional view of the endovascular brush in a retracted position being introduced into a sheath of the cell sampling device taken along line 2-2 of FIG. 1A.

FIG. 3 is a side cross-sectional view of the endovascular brush in a retracted position prior to entering a target artery.

FIG. 4 is a side cross-sectional view of the endovascular brush in an expanded position after entering the target artery to collect endothelial cells.

FIG. 5 is a flow chart setting forth the steps of a method for sampling endothelial cells within a pulmonary vasculature.

### DETAILED DESCRIPTION

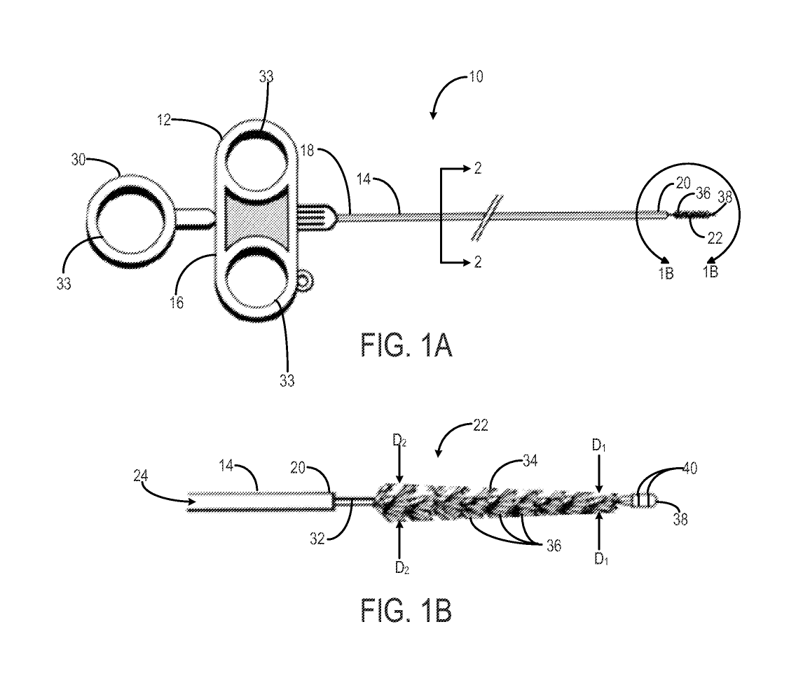

Referring to FIGS. 1A and 1B, a cell sampling device 10 according to one embodiment of the present disclosure is shown. The cell sampling device 10 may be utilized to sample pulmonary artery endothelial cells for endothelial phenotypic profiling in PAH and other forms of pulmonary hypertension. The cell sampling device 10 includes forceps 12 adapted for use internally of the body, for example in connection with vascular procedures. The forceps 12 include an elongated sheath 14 for introduction into the body and navigation to an area of interest. The forceps 12 include a control handle 16 at a proximal end 18 coupled to the elongated sheath 14 which extends over the main length of the cell sampling device 10. At a distal end 20, an endovascular brush 22 may extend from the elongated sheath 14 in an expanded position, as will be described in further detail below.

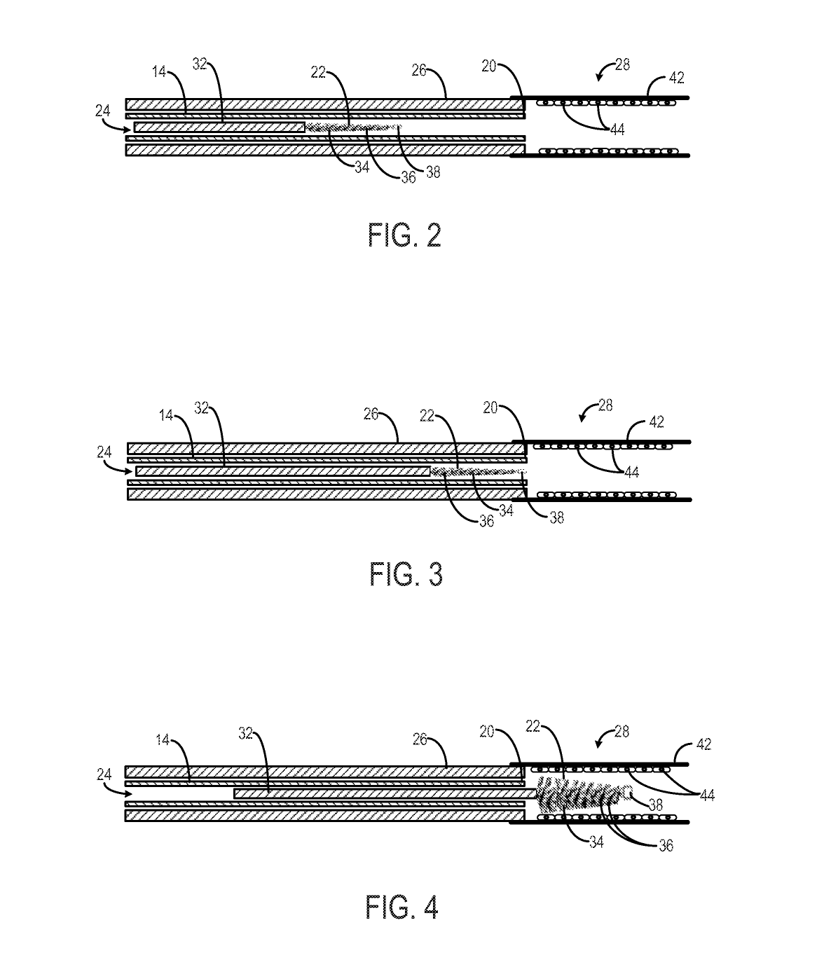

As shown in FIG. 2, the elongated sheath 14 may be a tubular member that defines an access lumen 24 extending from the proximal end 18 to the distal end 20. The elongated sheath 14 may receive at least a portion of the endovascular brush 22, which may be inserted into the elongated sheath 14 in preparation for catheterization. The elongated sheath 14 may vary dimensionally to accommodate various endovascular brush sizes. In one embodiment, the elongated sheath 14 is approximately 120 cm long in order to cover the entire length of the endovascular brush 22, and has an inner diameter of about 1.8 mm. In one non-limiting example, the elongated sheath 14 may be constructed of a transparent or semitransparent material so that the endovascular brush is visible through the elongated sheath 14. In addition, the elongated sheath 14 may be small enough such that it can be inserted into a catheter 26, such as a 5F or 6F coronary artery guiding catheter, a 7F myocardial biopsy sheath, or any suitable guiding sheath to allow the elongated sheath 14 to be delivered to a pulmonary vasculature 28.

Referring again to FIG. 1A, the forceps 12 include the control handle 16 and an actuation control 30. The actuation control 30 may be coupled to a shaft 32 of the endovascular brush 22, as shown in FIG. 2, such that movement of the actuation control 30 results in movement of the endovascular brush 22 within the access lumen 24. The actuation control 30 can include any type of actuating mechanism capable of imparting bidirectional axial movement to the shaft 32 within the elongated sheath 14. Loops 33 are provided in the control handle 16 and the actuation control 30 to form finger holes useful in grasping and manipulating the forceps 12.

Referring now to FIG. 1B, the endovascular brush 22 includes a brush segment 34, at a distal end of the endovascular brush 22, and the shaft 32 extending from the brush segment 34 to the proximal end of the endovascular brush 22. The brush segment 34 includes a plurality of brush elements 36 configured to retain sampled cells on the brush segment 34. The plurality of brush elements 36 may be abrasive elements that allow a user to remove tissue, such as endothelial cells, from the pulmonary vasculature 28 by rubbing or grazing an intravascular surface with the plurality of brush elements 36. Various types of brush elements 36 may be used to provide such functionality, including fibers, bristles, ridges, corrugations, and the like.

The endovascular brush 22 may further include a rounded tip 38, such as a spherical or semispherical tip, to inhibit injury to the vascular tissue during catheterization and/or sampling of endothelial cells. In one non-limiting example, the rounded tip 38 may include one or more radiopaque markers 40 at the distal end, or be made of a radiopaque material, allowing at least a portion of the endovascular brush 22 to be imaged for intraoperative image guidance. For example, the radiopaque markers 40 may be used to mark the intravascular location of the endovascular brush 22 of the cell sampling device 10 with fluoroscopy.

With continued reference to FIG. 1B, the plurality of brush elements 36 may be configured about the brush segment 34 in a variety of ways and still achieve the desired functionality. In one non-limiting example, the plurality of brush elements 36 may be disposed radially about the shaft 32 to form a conical or tapered shape. The tapered shape of the brush elements 36 may allow the endovascular brush 22 to extend into vessels with progressively narrower diameters and to ensure contact with the vessel wall, for example. The tapered brush elements 36 may have a distal diameter D₁ ranging from about 1 mm to about 3 mm, and a proximal diameter D₂ ranging from about 2.5 mm to about 5 mm. Other alternatives for the shape of the brush elements 36 include, but are not limited to, the incorporation of a curve in the brush, flat, one side flat or curved with a cylindrical inflatable balloon attached to the other side, helical, fan, oval, square, triangular, octagonal, or rectangular.

Rapid blood flow in human arteries can easily wash away the endothelial or other target cells that have been dislodged from the vessel wall by the cell sampling device 10. Accordingly, the plurality of brush elements 36 may be configured to retain the sampled endothelial cells in the presence of the moving fluid. In one embodiment, the size, shape, surface texture, spacing of the brush elements (e.g., the fibers or bristles) or a combination thereof are configured to provide a space or spaces where focal blood flow is relatively slow, which reduces the likelihood of target cells from being washed away in the fluid flow and correspondingly increases the likelihood for dislodged cells to remain on the brush elements 36. The brush elements 36, including fibers, bristles, or other elements may be modified to incorporate cell-specific antibodies to capture and retain cells or coated with a biological that will facilitate cell adhesion.

Additionally, thrombogenicity may be tailored or reduced in various ways. For example, the brush elements 36 may be constructed from a non-thrombogenic material, such as platinum, or may be formed from a thrombogenic material coated or impregnated with a non- or antithrombogenic substance or agent, such as heparin, hirudin, and the like, to reduce the material's thrombogenicity. This aspect reduces thrombus formation as a result of contact between the brush elements 36 and blood. The brush elements 36 may be constructed of made of polyurethane, polyester, polyglycolic acid, PFTE, polypropylene, polyethylene, nylon, rayon, or Dacron, for example, to provide a softer bristle that is less likely to cause intimal dissection, vessel perforation or other type of vascular injury. In some embodiments, the configuration of the brush elements 36 may also vary and can be arranged in a homogeneous manner or contain open or closed areas, be wavy, bent, straight, or folded in different patterns, for example.

In one non-limiting example, the brush segment 34 may be relatively flexible and elastic such that the brush segment 34 may be flexed, bent, turned, bowed, twisted, stretched, compressed, or otherwise deformed, from an original shape, without breaking, and capable of recovering the original shape from the deformed shape. This flexibility of the brush segment 34 may be achieved, for example, by forming the brush segment 34 from a material having suitable flexibility and elasticity so that the brush segment 34 returns to the original shape upon removal of the deforming forces.

With continued reference to FIG. 1B, the shaft 32 of the endovascular brush 22 may be made of various materials and in various lengths. The shaft 32 should generally be of sufficient length to access the pulmonary vasculature 28 from a selected point of entry. In one embodiment, the shaft 32 may be constructed of a stainless steel alloy, titanium, nitinol, or nickel alloys, for example.

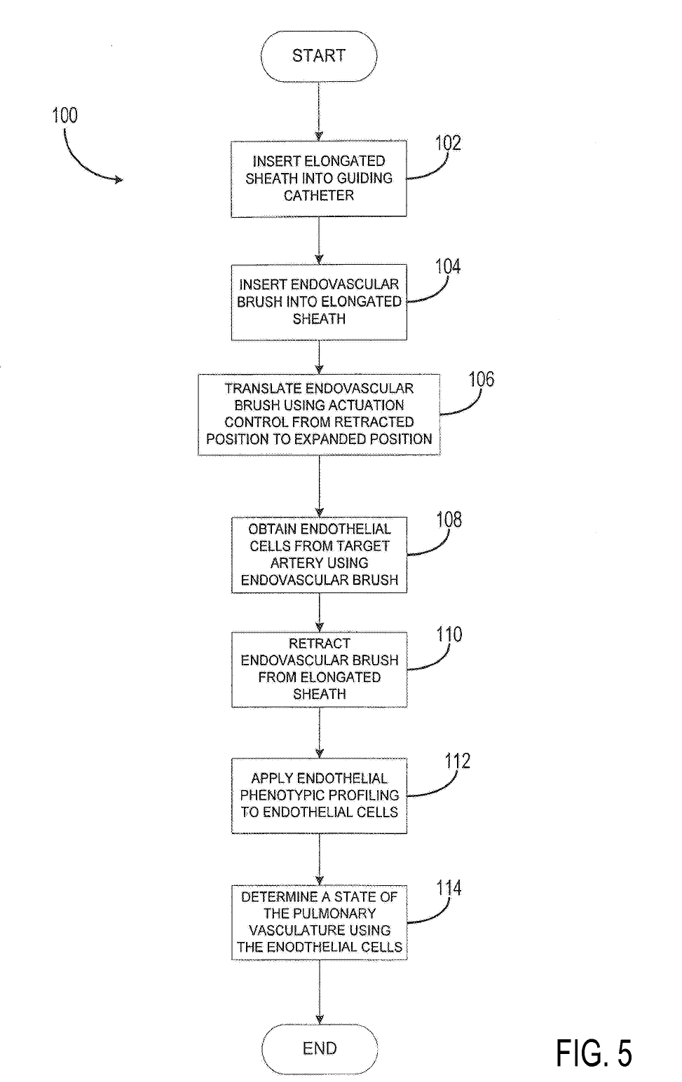

Referring now to FIG. 5, a flow chart setting forth exemplary steps 100 for sampling endothelial cells from arteries and veins within a subject's pulmonary vasculature 28 is provided. To start the process, at process block 102, the elongated sheath 14 may be inserted into the guiding catheter 26 that has been inserted into the a target artery 42 (see FIG. 2), proximal to the intended site of interest, such as at a vascular site have a lesion thereon. The elongated sheath 14 may be inserted into the guiding catheter 26 until the distal end 20 of the elongated sheath 14 engages a distal end of the catheter 26, as shown in FIG. 2. Next, at process block 104, a user may insert the endovascular brush 22 into the access lumen 24 of the elongated sheath 14, such that the endovascular brush 22 is in a retracted position, as shown in FIG. 2.

At process block 106, the actuation control 30 of the forceps 12 may be used to axially translate the endovascular brush 22 from the retracted position to the expanded position, as shown in FIG. 4. Activation of the actuation control 30 may push the shaft 32 linearly through the access lumen 24 of the elongated sheath 14, thereby advancing the brush elements 36 into the elongated sheath 14 toward the target artery 42, as shown in FIG. 3. In one non-limiting example, the endovascular brush 22 may be inserted through the subject's right ventricular apex and advanced to the pulmonary artery under direct visualization, such as through biplane fluoroscopic guidance and Ultravist 370 contrast imaging.

As shown in FIG. 4, the actuation control 30 may be used to push the brush elements 36 out from the elongated sheath 14 to expose the brush elements 36 in the target artery 42. Next, at process block 108, the brush elements 36 may be used to obtain endothelial cells 44 from the target artery 42. Once inserted into the target artery 42, the brush elements 36 may expand and provide the necessary pressure against the target artery 42 to remove endothelial cells 44 there from. In one non-limiting example, the location of the brush elements 36 in relation to the target artery 42 may be confirmed by imaging the site of interest, for example, with x-ray imaging, fluoroscopy, and/or contrast injection if necessary, to ensure that the brush elements 36 come into contact or overlap the intended vascular segment or lesion.

The endothelial cells 44 may then be sampled from the target artery 42 by pulling the actuation control 30 in a swift motion, which causes the shaft 32 to retract, and the brush elements 36 to rub against or scrape the target artery 42, and capture the endothelial cells 44 there from. Then, at process block 110, the endovascular brush 22 may be retracted into the catheter 26 and into the access lumen 24 of the elongated sheath 14, as shown in FIG. 2. In some embodiments, at process block 112, endothelial phenotypic profiling may be applied to the captured endothelial cells 44 to determine a state of the pulmonary vasculature at process block 114. For example, the state of the pulmonary vasculature may indicate the presence of PAH.

Additionally, or alternatively, once the cell sampling device 10 is removed from the subject, the brush elements 36 of the endovascular brush 22 may be immersed in dissociation medium to collect the cells. The dissociation medium may then be centrifuged and the isolated endothelial cells 44 may be plated on a chamber slide and allowed to adhere. The cells may then be assessed for endothelial origin by immunofluorescent staining for CD31, or allowed to expand in culture.

In one non-limiting example, to evaluate the efficacy of the cell sampling device 10 to sample pulmonary artery endothelial cells for phenotypic analysis by genome array expression profiling, the endothelial cells retrieved may be quantified using a hemocytometer. A 10 μl aliquot of suspended cells may be quantified by the hemocytometer and a total number determined by the average count per square multiplied by the dilution factor and multiplied by 10⁴. If the number of cells retrieved is insufficient to obtain enough RNA for microarray analysis, the cells may be plated in a P25 tissue culture dish, for example, and expanded. Briefly, cells may be placed in endothelial cell medium (Lonza) with 2.5% fetal bovine serum. Once the plate is confluent, the cells may be harvested for RNA isolation. The percentage of cells that are true endothelial cells may be determined using immunofluorescence immunohistochemistry to detect CD31 expression.

To characterize endothelial gene expression, RNA may be isolated from cells using, for example, an RNEasy Minikit (Qiagen) and endothelial phenotype may be analyzed using a gene expression microarray that contains approximately 43,603 oligonucleotide probes. cDNA may be labeled using cyanine-3-CTP and hybridized with the microarray slide at approximately 65° C. for about 17 hours. The slides may then be washed according the manufacturer's instructions and scanned with, for example, an Agilent DNA Microarray Scanner. The images may be analyzed using Agilent Feature Extraction Software, and data may be normalized and functional gene categories may be assigned using the annotation, visualization and integrated discovery (DAVID) bioinformatics database.

For each time-point that the endothelial cells are harvested, an endothelial phenotypic profile may be defined by a panel of up to 5 genes that show the greatest differential expression between a sham and pulmonary hypertension subjects. These genes may be validated using standard qRT-PCR methodology (ABI Biosystems). Preference may be given to genes that are also differentially expressed over time in the pulmonary hypertension model. The endothelial (patho) phenotypes may then be compared with the hemodynamic variables to identify patterns of gene expression that predict or correlate with hemodynamic indices of pulmonary hypertension.

In yet another non-limiting example, the endovascular brush 22 may be by evaluated by assessing the pulmonary artery using angiography, histology, and electron microscopy to verify injury and/or damage to the pulmonary artery was minimal. After user of the endovascular brush 22, selective pulmonary angiography may be used to determine if there has been a perforation (e.g., leakage of contrast dye), a dissection (e.g., “hang-up” of contrast dye outside the vessel), thrombus formation in situ (e.g., intravascular dye stain with filling defect), or vessel occlusion (e.g., abrupt cut-off with dye “hang-up”).

To perform the angiogram, the endovascular brush 22 may be removed from the guiding catheter 26 and a SF pigtail catheter, for example, may be advanced over a wire to the recently sampled pulmonary artery and placement confirmed using biplane angiography. A 20 cc injection of Ultravist 370 contrast may be performed using a power injector and recorded as a cine angiogram. Angiograms may be reviewed by one or more operators to verify the absence of any vascular injury patterns.

The present disclosure has been described in terms of one or more preferred embodiments, and it should be appreciated that many equivalents, alternatives, variations, and modifications, aside from those expressly stated, are possible and within the scope of the disclosure.

1. A cell sampling device for sampling endothelial cells within a pulmonary vasculature, the cell sampling device comprising:
an elongated sheath extending from a proximal end to a distal end of the cell sampling device and defining an access lumen;
an endovascular brush including brush elements coupled to a shaft, the endovascular brush dimensioned to be received in the access lumen of the elongated sheath;
an actuation control coupled to the shaft of the endovascular brush to axially translate the endovascular brush between a retracted position and an expanded position; and
wherein upon translation of the endovascular brush from the retracted position to the expanded position, the brush elements move from within the access lumen to the pulmonary vasculature to obtain endothelial cells from a target artery of the pulmonary vasculature.

2. The cell sampling device of claim 1, further comprising a control handle coupled to the proximal end of the elongated sheath, wherein the actuation control is configured to move linearly toward the control handle to move the endovascular brush to the expanded position.

3. The cell sampling device of claim 2, wherein at least one of the control handle or the actuation control includes a loop sized to receive a user's finger to manipulate the cell sampling device.

4. The cell sampling device of claim 1, wherein the elongated sheath is dimensioned to be received by a guiding catheter inserted into the target artery to deliver the elongated sheath and the endovascular brush to the pulmonary vasculature.

5. The cell sampling device of claim 1, wherein the elongated sheath includes a tubular member constructed from at least one of a transparent or semitransparent material for identifying a location of the brush elements within the access lumen relative to the target artery.

6. The cell sampling device of claim 1, wherein the brush elements are configured to retain the endothelial cells when engaged with the target artery.

7. The cell sampling device of claim 1, wherein the brush elements include at least one of fibers, bristles, ridges or corrugations.

8. The cell sampling device of claim 1, wherein the brush elements are disposed radially about the shaft to form at least one of a conical or tapered shape to allow the endovascular brush to extend into the target artery.

9. The cell sampling device of claim 8, wherein the at least one of the conical or tapered shape includes a distal diameter ranging from about 1 mm to about 3 mm and a proximal diameter ranging from about 2.5 mm to about 5 mm.

10. The cell sampling device of claim 1, wherein the brush elements are disposed about the shaft in at least one of a homogenous manner, a non-homogeneous manner, a wavy manner, a bent manner, or a folded manner to allow the endovascular brush to extend into the target artery.

11. The cell sampling device of claim 1, wherein the brush elements are disposed about the shaft to form at least one of a curved shape, a flat shape, a helical shape, a fan shape, an oval shape, a square shape, a triangular shape, an octagonal shape, or a rectangular shape.

12. The cell sampling device of claim 1, wherein the brush elements are disposed about the shaft to form at least one of a flat shape or a curved shape on a first side of the shaft and the shaft including a cylindrical inflatable balloon coupled to a second side of the shaft.

13. The cell sampling device of claim 1, wherein the brush elements are constructed from at least one of a non-thrombogenic material or a thrombogenic material including an antithrombogenic substance to reduce thrombogenicity.

14. The cell sampling device of claim 1, wherein the elongated sheath includes a length of about 120 cm to cover a length of the endovascular brush and an inner diameter of about 1.88 mm.

15. The cell sampling device of claim 1, wherein the endovascular brush includes at least one of a spherical or semispherical tip to inhibit injury to the target artery when the endovascular brush is in the expanded position.

16. The cell sampling device of claim 15, wherein the at least one spherical or semispherical tip includes at least one radiopaque marker to allow the endovascular brush to be imaged with fluoroscopy to identify a location of the endovascular brush relative to the target artery.

17. A method for sampling endothelial cells within a pulmonary vasculature, the method comprising:
inserting an elongated sheath into the pulmonary vasculature of a subject, the elongated sheath defining an access lumen;
inserting an endovascular brush into the access lumen of the elongated sheath, the endovascular brush including brush elements coupled to a shaft;
using an actuation control coupled to the shaft of the endovascular brush to axially translate the endovascular brush from a retracted position within the access lumen to an expanded position in a target artery of the pulmonary vasculature; and
obtaining, using the brush elements of the endovascular brush, endothelial cells from the target artery to determine a state of the pulmonary vasculature.

18. The method of claim 17, wherein using the actuation control to axially translate the endovascular brush to the expanded position in the target artery includes translating the actuation control relative to a control handle coupled to a proximal end of the elongated sheath.

19. The method of claim 17, further comprising guiding the elongated sheath into a guiding catheter inserted into the target artery to deliver the elongated sheath and the endovascular brush to the pulmonary vasculature.

20. The method of claim 17, further comprising:
applying endothelial phenotypic profiling to the endothelial cells to determine if the state of the pulmonary vasculature includes at least one of pulmonary arterial hypertension or other forms of pulmonary hypertension;
identifying a state of the at least one of pulmonary arterial hypertension or other forms of pulmonary hypertension; and
assessing a response to medical therapy.

21. The method of claim 17, wherein the endovascular brush includes at least one of a spherical or semispherical tip to inhibit injury to the target artery when the endovascular brush is in the expanded position.

22. The method of claim 21, wherein the at least one spherical or semispherical tip includes at least one radiopaque marker to allow the endovascular brush to be imaged with fluoroscopy to identify a location of the endovascular brush relative to the target artery.

23. The method of claim 17, wherein the brush elements are disposed radially about the shaft to form at least one of a conical or tapered shape to allow the endovascular brush to extend into the target artery.
